(19) United States
(12) Patent Application Publication
(10) Pub. No.: US 2002/0127692 A1
(43) Pub. Date: Sep. 12, 2002

(54) Protein

(76) Inventors: Barbara S. Ink, Stevenage (GB); Alan Peter Lewis, Stevenage (GB)

Correspondence Address: DAVID J LEVY, CORPORATE INTELLECTUAL PROPERTY, GLAXOSMITHKLINE, FIVE MOORE DR., PO BOX 13398, DURHAM, NC, 27709-3398, US

(21) Appl. No.: 10/008,461

(22) Filed: Nov. 13, 2001

(30) Foreign Application Priority Data
Nov. 15, 2000 (GB) 0027905.9

(51) Int. Cl. C12N 9/64; C12Q 1/68; C07H 21/04; C12P 21/02; C12N 5/06

(52) U.S. Cl. 435/226; 435/6; 435/69.1; 435/325; 435/320.1; 536/23.2

(57) ABSTRACT

The present invention provides an isolated cysteine proteinase polypeptide comprising

(i) the amino acid sequence of SEQ ID NO: 2; or

(ii) a variant thereof which is capable of cleaving SUMO from a target protein and/or cleaving the precursor form of SUMO to release the active form of SUMO; or

(iii) a fragment of (i) or (ii) which is capable of cleaving SUMO from a target protein and/or cleaving the precursor form of SUMO to release the active form of SUMO.

### CROSS REFERENCE TO RELATED APPLICATIONS

This application claims priority from Great Britain application number 0027905.9 filed on Nov. 15, 2000.

### FIELD OF THE INVENTION

The present invention relates to cysteine proteinase polypeptides.

### BACKGROUND OF THE INVENTION

There are 4 main catalytic types of peptidases; serine (which includes threonine peptidases) cysteine, aspartic and metallo. The serine, threonine and cysteine peptidases are catalytically very different from the aspartic and metallopeptidases in that the nucleophile of the catalytic site is part of an amino acid, whereas it is an activated water molecule in the other groups.

Peptidases in which the nucleophile is the sulphydryl group of a cysteine residue are known as cysteine-type peptidases. The catalytic mechanism is similar to that of the serine-type peptidases in that a nucleophile and a proton donor or general base is required, and the proton donor in all cysteine peptidases (in which it has been identified) is a histidine residue. Typical examples of cysteine peptidases include the cathepsins (excluding cathepsin G) and caspases.

Human ubiquitin-like protein specific protease (ULP). Ulp is a deconjugating enzyme of SUMO/Sentrin, a ubiquitin-like protein, and its target protein. It is a cysteine protease that is unrelated to other known deubiquitinating enzymes, but shows similarity to certain viral proteases. The Ulps cleave at a glycine-glycine-X cleavage site that is similar to the consensus sites of adenovirus, African swine fever virus, and certain poxviruses. Ulps also cleave the SUMO precursor to generate the mature form of SUMO.

The human ULPs show homology to the yeast ULP1 and ULP2 genes. Yeast ULP1 cleaves the yeast homolog of SUMO, SMT3, from target proteins. ULP1 is important throughout the cell cycle and is essential for the G2/M phase. ULP2 also cleaves SMT3 from target proteins, although the target proteins of ULP2 appear to be different from ULP 1. ULP2 is important for the recovery of cells from checkpoint arrest induced by DNA damage, inhibition of DNA replication, or defects in spindle assembly. Deletions of ULP2 in yeast show a phenotype of temperature-sensitive growth, abnormal cell morphology, decreased plasmid and chromosome stability, and a severe sporulation defect.

### SUMMARY OF THE INVENTION

A novel cysteine proteinase, referred to herein as HIPHUM 119, is now provided. HIPHUM 119 is shown to be expressed in all tissues at various levels. It is highly expressed in adrenal, cerebellum, rectum, testis, thyroid and urinary bladder. It is also expressed at significant levels in lung, fetal brain, skeletal muscle, tonsil and uterus. In addition, it is expressed in T cells, peripheral blood mononucleocytes (PBMNCs), monocytes and dendritic cells. The novel cysteine proteinase is a screening target for the identification and development of novel pharmaceutical agents, including modulators of cysteine proteinase activity. These agents may be used in the treatment and/or prophylaxis of disorders such as HIV infection, lung cancer, inflammatory disease such as asthma, Hepatitis B and brain diseases such as Alzheimer's disease, parasupranuclear palsey and Huntington's disease.

Accordingly, the present invention provides an isolated cysteine proteinase polypeptide comprising:

(i) the amino acid sequence of SEQ ID NO: 2;

(ii) a variant thereof which is capable of cleaving SUMO from a target protein and/or cleaving the precursor form of SUMO to release the active form of SUMO; or

(iii) a fragment of (i) or (ii) which is capable of cleaving SUMO from a target protein and/or cleaving the precursor form of SUMO to release the active form of SUMO.

According to another aspect of the invention there is provided a polynucleotide encoding a polypeptide of the invention which polynucleotide includes a sequence comprising:

(a) the nucleic acid sequence of SEQ ID NO: 1 and/or a sequence complementary thereto;

(b) a sequence which hybridises under stringent conditions to a sequence as defined in (a);

(c) a sequence that is degenerate as a result of the genetic code to a sequence as defined in (a) or (b); or

(d) a sequence having at least 60% identity to a sequence as defined in (a), (b) or (c).

The invention also provides:

an expression vector which comprises a polynucleotide of the invention and which is capable of expressing a polypeptide of the invention;

a host cell comprising an expression vector of the invention;

a method of producing a polypeptide of the invention which method comprises maintaining a host cell of the invention under conditions suitable for obtaining expression of the polypeptide and isolating the said polypeptide;

an antibody specific for a polypeptide of the invention;

a method for identification of a substance that modulates cysteine proteinase activity and/or expression, which method comprises contacting a polypeptide, polynucleotide, expression vector or host cell of the invention with a test substance and determining the effect of the test substance on the activity and/or expression of the said polypeptide or the polypeptide encoded by the said polynucleotide, thereby to determine whether the test substance modulates cysteine proteinase activity and/or expression;

a compound which or modulates cysteine proteinase activity and which is identifiable by the method referred to above;

a method of treating a subject having a disorder that is responsive to cysteine proteinase stimulation or modulation, which method comprises administering to said subject an effective amount of substance of the invention; and

use of a substance that stimulates or modulates cysteine proteinase activity in the manufacture of a medicament for the treatment or prophylaxis of a disorder that is responsive to stimulation or modulation of cysteine proteinase activity.

Preferably the disorder is selected from HIV infection, lung cancer, inflammatory disease such as asthma, Hepatitis B and brain diseases such as Alzheimer's disease, parasupranuclear palsey and Huntington's disease.

### BRIEF DESCRIPTION OF THE FIGURES

FIG. 1 shows the relative expression levels of HIPHUM 119 in human tissues.

FIG. 2 shows the relative expression of HIPHUM 119 in normal and tumor tissue from the colon, lung and breast.

FIG. 3 shows the relative expression of HIPHIUM 119 in a normal brain and brain tissue from Alzheimer's disease (Alz), Huntington's disease (Hunt), myotonic dystrophy (MD), parasupranuclear palsey (PSP) and ALS.

FIG. 4 shows the relative expression of HIPHUM 119 in normal lung and tissue from asthmatic and chronic obstructive pulmonary disease (COPD) lung; in control endothelial cells, VEGF treated endothelial cells and bFGF treated endothelial cells; in stimulated and unstimulated bone marrow; in normal knee cartilage and cartilage from osteoarthritic knee; in HS-1 and HS-2 synovium in rheumatoid arthritis; and in differentiated and undifferentiated osetoclasts.

FIG. 5 shows the relative expression levels of HIPHUM 119 in various blood cells and in HSV, HBV and HIV/PBL infected cells.

### BRIEF DESCRIPTION OF THE SEQUENCES

SEQ ID NO: 1 shows the nucleotide and amino acid sequences of human protein HIPHUM 119.

SEQ ID NO: 2 is the amino acid sequence alone of HIPHUM 119.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a human cysteine proteinase, referred to herein as HIPHUM 119, and variants thereof. Sequence information for HIPHUM 119 is provided in SEQ ID NO: 1 (nucleotide and amino acid) and in SEQ ID NO: 2. A polypeptide of the invention thus consists essentially of the amino acid sequence of SEQ ID NO: 2 or of a variant of that sequence, or of a fragment of either thereof.

Polypeptides of the invention may be in a substantially isolated form. It will be understood that the polypeptide may be mixed with carriers or diluents which will not interfere with the intended purpose of the polypeptide and still be regarded as substantially isolated. A polypeptide of the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention. Routine methods, can be employed to purify and/or synthesise the proteins according to the invention. Such methods are well understood by persons skilled in the art, and include techniques such as those disclosed in Sambrook et al, Molecular Cloning: a Laboratory Manual, 2^{nd} Edition, CSH Laboratory Press, 1989, the disclosure of which is included herein in its entirety by way of reference.

The term “variant” refers to a polypeptide which has a same essential character or basic biological functionality as HIPHUM 119. The essential character of HIPHUM 119 can be defined as follows: HIPHUM 119 is a cysteine proteinase. Preferably a variant polypeptide is one which binds to the same a target protein as HIPHUM 119. Preferably the polypeptide is capable of cleaving SUMO from a target protein and/or cleaving the precursor form of SUMO to release the active form of SUMO. Preferably the polypeptide is capable of cleaving the SUMO precursor to its mature and active form.

Preferably the polypeptide comprises a catalytic domain which comprises a catalytic triad. The catalytic triad is typically composed of a cysteine residue, histidine residue, aspartate residue and a glutamine residue which together form the oxyanion hole at the active site. Preferably the polypeptide comprises an endoplasmic reticulum (ER) retention signal. Preferably the ER retention signal comprises the four amino acids, TKKR. Preferably the polypeptide comprises a leucine zipper pattern. Preferably the leucine zipper pattern is LSYMDSLLRQSDVSLLDPPSWL.

A polypeptide having a same essential character as HIPHUM 119 may be identified by monitoring for a function the cysteine proteinase selected from cleavage of SUMO from a target protein, activation of SUMO and alteration in activity or subcellular localisation of a target protein. Typical target proteins include transcription factors, such as IκBα/NF-κB, p53, c-jun and HIPK2, proteins involved in intracellular transport, such as RanGAP1, GLUT1 and GLUT4, proteins involved in the intracellular response to DNA damage and repair, such as Topoisomerase 1, Topoisomerase 2, MDM2 and WRN, proteins involved in nuclear localisation, such as PML and Sp100, and viral proteins such as bovine papillomavirus E1 and human cytomegalovirus E2. Other target proteins may be identified, for example using the yeast-2-hybrid assay. Such target proteins that have been identified using the yeast-2-hybrid screen include TNF and FAS receptors, CEN, and rad 52.

SUMO binds to various transcription factors and is involved in their regulation. Removal of SUMO from IKBA would allow for activation of NF-κB and produce an inflammatory response. SUMO conjugation increases the transcriptional activity of p53, therefore, deconjugation of SUMO may lower the transcriptional activity of p53. C-jun modification by SUMO decreases its transcriptional activity. Therefore, Ulp proteases may regulate tumorsuppressor activity with a role in cell cycle arrest, apoptosis, and cellular responses to mitogens, stress, or inflammatory stimuli.

SUMO conjugates to a nuclear kinase, HIPK2, and is needed for the formation of nuclear bodies containing HIPK2. HIPK2 can act as a transcriptional corepressor for homeoproteins. Therefore, deconjugation of SUMO by the protease may regulate the activity of homeodomain transcription factors and play a role in development.

SUMO modification of RanGAP1 targets the complex to the nuclear pore complex from the cytosol. Ulp protease may be involved in the regulation of the levels of the Ran GAP1 within the cytosol and pore complex.

SUMO also modifies GLUT1 and GLUT4 that are involved in insulin stimulated glucose uptake. Deconjugation of SUMO would regulate the amount of glucose uptake in the cell.

The Ulp protease may regulate the cellular response to DNA damage and DNA repair. SUMO conjugates to TOP1 and TOP2 when treated with DNA damaging agents. MDM2 is also conjugated to SUMO to prevent self-ubiquitination of mdm2, thus enhancing E3 ligase activity of p53. A Ulp protease may regulate the conjugation of SUMO to MDM2, thereby affecting stability of p53. SUMO also conjugates to WRN that functions as a DNA helicase, exonuclease, and ATPase. Deletions of WRN which are found in Werner's syndrome impair its nuclear localization. It is possible that cleavage of SUMO from WRN would alter the localization of WRN and contribute to its dysregulation during disease.

SUMO modification of PML is needed for its localization into nuclear bodies containing DAXX. PML is involved in many cellular functions such as the interferonresponse, immune surveillance, apoptosis, and as a tumor suppressor. It appears that the function of PML is to meditate the regulation of transcription. Mice lacking PML have shown altered transcription in cellular differentiation by way of nuclear receptors, apoptosis mediated by DAXX and p53, inhibition of growth mediated by pRb and p53, and the immune response through interferons. Therefore, regulating the conjugation-deconjugation of SUMO to PML through Ulp protease may affect these pathways.

Although the function of the interferon-induced protein, Sp100, is unknown, conjugation to SUMO takes place in the nucleus. The Ulp protease may affect the activity of Sp100 in response to interferon.

SUMO conjugates to the human cytomegalovirus protein, IE2. IE2 functions as a strong transactivator of both viral and cellular promoters. Overexpression of SUMO reduces the transactivation activity of IE2. Therefore, the Ulp protease may function to control the level of IE2 sumoylation, thus, affecting both the virus life cycle and host interactions to virus infection.

SUMO conjugates to the bovine papalomavirus E1 protein which functions as the iniatiator of replication. Mutants which cannot conjugate SUMO are not able to localise to the correct nuclear subdomain. The Ulp protease may control the level of sumylation of E1, thus affecting both the virus life cycle and host reactions to viral infection.

Preferably a polypeptide of the invention is located in the endoplasmic reticulum when expressed in a cell. More preferably a polypeptide of the invention is capable of cycling between the endoplasmic reticulum and other intracellular compartments.

In another aspect of the invention, a variant is one which does not show the same activity as HIPHUM 119 but is one which inhibits a basic function of HIPHUM 119. For example, a variant polypeptide is one which inhibits protease activity of HIPHUM 119, for example by binding to a target protein to prevent a target protein binding to HIPHUM 119.

Typically, polypeptides with more than about 65% identity preferably at least 80% or at least 90% and particularly preferably at least 95% at least 97% or at least 99% identity, with the amino acid sequences of SEQ ID NO: 2, are considered as variants of the proteins. Such variants may include allelic variants and the deletion, modification or addition of single amino acids or groups of amino acids within the protein sequence, as long as the peptide maintains a basic biological functionality of the HIPHUM 119 receptor.

Amino acid substitutions may be made, for example from 1, 2 or 3 to 10, 20 or 30 substitutions. The modified polypeptide generally retains activity as a cysteine proteinase. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Shorter polypeptide sequences are within the scope of the invention. For example, a peptide of at least 20 amino acids or up to 50, 60, 70, 80, 100, 150, 200, 300 or 400 amino acids in length is considered to fall within the scope of the invention as long as it demonstrates a basic biological functionality of HIPHUM 119. In particular, but not exclusively, this aspect of the invention encompasses the situation when the protein is a fragment of the complete protein sequence and may represent a catalytic domain or substrate binding domain. Preferred fragments include the C-terminal catalytic domain, the N-terminal domain which is not conserved between different members of the Ulp family of proteases, the leucine zipper domain or the ER membrane retention signal. Such fragments can be used to construct chimeric proteases preferably with another protease, more preferably with another member of the family of cysteine proteinases. Such chimeric proteases may comprise different domains from different cysteine proteinases. For example, a fragment comprising an N-terminal domain of a polypeptide of the invention may be fused to a C-terminal catalytic domain of a different cysteine proteinase.

Fragments of HIPHUM 119 or a variant thereof can also be used to raise anti-HIPHUM 119 antibodies. In this embodiment the fragment may comprise an epitope of the HIPHUM 119 polypeptide and may otherwise not demonstrate the catalytic, substrate binding or other properties of HIPHUM 119.

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

The invention also includes nucleotide sequences that encode for HIPHUM 119 or variant thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1. Such nucleotides can be isolated from human cells or synthesised according to methods well known in the art, as described by way of example in Sambrook et al, 1989.

Typically a polynucleotide of the invention comprises a contiguous sequence of nucleotides which is capable of hybridizing under selective conditions to the coding sequence or the complement of the coding sequence of SEQ ID NO: 1.

A polynucleotide of the invention can hydridize to the coding sequence or the complement of the coding sequence of SEQ ID NO: 1 at a level significantly above background. Background hybridization may occur, for example, because of other cDNAs present in a cDNA library. The signal level generated by the interaction between a polynucleotide of the invention and the coding sequence or complement of the coding sequence of SEQ ID NO: 1 is typically at least 10 fold, preferably at least 100 fold, as intense as interactions between other polynucleotides and the coding sequence of SEQ ID NO: 1. The intensity of interaction may be measured, for example, by radiolabelling the probe, e.g. with ³²P. Selective hybridisation may typically be achieved using conditions of medium to high stringency. However, such hybridisation may be carried out under any suitable conditions known in the art (see Sambrook et al, 1989. For example, if high stringency is required suitable conditions include from 0.1 to 0.2× SSC at 60° C. up to 65° C. If lower stringency is required suitable conditions include 2× SSC at 60° C.

The coding sequence of SEQ ID NO: 1 may be modified by nucleotide substitutions, for example from 1, 2 or 3 to 10, 25, 50 or 100 substitutions. The polynucleotide of SEQ ID NO: 1 may alternatively or additionally be modified by one or more insertions and/or deletions and/or by an extension at either or both ends. A polynucleotide may include one or more introns, for example may comprise genomic DNA. Additional sequences such as signal sequences which may assist in insertion of the polypeptide in a cell membrane may also be included. The modified polynucleotide generally encodes a polypeptide which has a HIPHUM 119 activity. Alternatively, a polynucleotide encodes a catalytic or substrate-binding portion of a polypeptide or a polypeptide which inhibits HIPHUM 119 activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1 will generally have at least 60%, at least 70%, at least 80%, at least 90%, at least 95%, at least 98% or at least 99% sequence identity to the coding sequence of SEQ ID NO: 1 over a region of at least 20, preferably at least 30, for instance at least 40, at least 60, more preferably at least 100 contiguous nucleotides or most preferably over the full length of SEQ ID NO: 1.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques. Nucleotides complementary to those encoding HIPHUM 119, or antisense sequences, may also be used in gene therapy.

Polynucleotides of the invention may be used as a primer, e.g. a PCR primer, a primer for an alternative amplification reaction, a probe e.g. labelled with a revealing label by conventional means using radioactive or non-radioactive labels, or the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500 or 600 nucleotides in length, or even up to a few nucleotides, such as five or ten nucleotides, short of the coding sequence of SEQ ID NO: 1.

The present invention also includes expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. 1989.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention or may be useful in a method of treatment of the human or animal body by therapy.

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistence gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

The invention also includes cells that have been modified to express the HIPHUM 119 polypeptide or a variant thereof. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably the cell line selected will be one which is not only stable, but also allows for mature glycosylation of a polypeptide. Expression may be achieved in transformed oocytes. A polypeptide of the invention may be expressed in cells of a transgenic non-human animal, preferably a mouse. A transgenic non-human animal expressing a polypeptide of the invention is included within the scope of the invention. A polypeptide of the invention may also be expressed in Xenopus laevis oocytes.

A polypeptide of the invention may be overexpressed in bacterial cells, such as E. coli, and isolated from the bacterial culture.

According to another aspect, the present invention also relates to antibodies, specific for a polypeptide of the invention. Such antibodies are for example useful in purification, isolation or screening methods involving immunoprecipitation techniques or, indeed, as therapeutic agents in their own right.

Antibodies may be raised against specific epitopes of the polypeptides according to the invention. Such antibodies may be used to block substrate binding to the receptor. An antibody, or other compound, “specifically binds” to a protein when it binds with preferential or high affinity to the protein for which it is specific but does substantially bind not bind or binds with only low affinity to other proteins. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

Antibodies of the invention may be antibodies to human polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample, which method comprises:

I providing an antibody of the invention;

II incubating a biological sample with said antibody under conditions which allow for the formation of an antibody-antigen complex; and

III determining whether antibody-antigen complex comprising said antibody is formed.

A sample may be for example a tissue extract, blood, serum and saliva. Antibodies of the invention may be bound to a solid support and/or packaged into kits in a suitable container along with suitable reagents, controls, instructions, etc. Antibodies may be linked to a revealing label and thus may be suitable for use in methods of in vivo HIPHUM 119 imaging.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An important aspect of the present invention is the use of polypeptides according to the invention in screening methods. The screening methods may be used to identify substances that bind to cysteine proteinase and in particular which bind to HIPHUM 119 such as a substrate for the enzyme. Screening methods may also be used to identify agonists or antagonists which may modulate cysteine proteinase activity, inhibitors or activators of HIPHUM 119 activity, and/or agents which up-regulate or down-regulate HIPHUM 119 expression.

Any suitable format may be used for the assay. In general terms such screening methods may involve contacting a polypeptide of the invention with a test substance and monitoring for binding of the test substance to the polypeptide or measuring protease activity. A polypeptide of the invention may be incubated with a test substance. Modulation of cysteine proteinase activity may be determined. In a preferred aspect, the assay is a cell-based assay. Preferably the assay may be carried out in a single well of a microtitre plate. Assay formats which allow high throughput screening are preferred.

A typical assay for determining whether a test substance acts as an inhibitor or activator of HIPHUM 119 activity comprises contacting a fluorescent or colourimetric substrate with a polypeptide of the invention and a test substance and monitoring protease activity by monitoring any change in the fluorescence or light emission. Any changes in the fluorescence of a substrate as a result of its proteolytic degradation by a polypeptide of the invention may be detected using a fluorescence plate reader. Colourimetic changes may be measured using a spectrophotometer. The inhibitory or stimulatory activity of a test substance may be determined by comparing any fluorescent or colourimetric changes observed in the presence of a test substance to any changes observed in the absence of a test substance and/or in the presence of a known inhibitor of HIPHUM 119 activity.

Modulator activity can be determined by contacting cells expressing a polypeptide of the invention with a substance under investigation and by monitoring an effect mediated by the polypeptide. The cells expressing the polypeptide may be in vitro or in vivo. The polypeptide of the invention may be naturally or recombinantly expressed. Preferably, the assay is carried out in vitro using cells expressing recombinant polypeptide. Preferably, control experiments are carried out on cells which do not express the polypeptide of the invention to establish whether the observed responses are the result of activation of the polypeptide.

The binding of a test substance to a polypeptide of the invention can be determined directly. For example, a radiolabelled test substance can be incubated with the polypeptide of the invention and binding of the test substance to the polypeptide can be monitored. Typically, the radiolabelled test substance can be incubated with cell membranes containing the polypeptide until equilibrium is reached. The membranes can then be separated from a non-bound test substance and dissolved in scintillation fluid to allow the radioactive content to be determined by scintillation counting. Non-specific binding of the test substance may also be determined by carrying out a competitive binding assay.

Substances that inhibit the interaction of a polypeptide of the invention with a HIPHUM 119 substrate or with another protease may also be identified through a yeast 2-hybrid assay or other protein interaction assay such as a co-immunoprecipitation or an ELISA based technique.

Assays may be carried out using cells expressing HIPHUM 119, and incubating such cells with the test substance optionally in the presence of a HIPHUM 119 substrate. The results of the assay are compared to the results obtained using the same assay in the absence of the test substance. Cells expressing HIPHUM 119 constitutively may be provided for use in assays for HIPHUM 119 function. Additional test substances may be introduced in any assay to look for inhibitors or activators of substrate binding or inhibitors or activators of protease activity.

Assays may also be carried out to identify substances which modify HIPHUM 119 expression, for example substances which up- or down-regulate expression. Such assays may be carried out for example by using antibodies for HIPHUM 119 to monitor levels of HIPHUM 119 expression. Other assays which can be used to monitor the effect of a test substance on HIPHUM 119 expression include using a reporter gene construct driven by the HIPHUM 119 regulatory sequences as the promoter sequence and monitoring for expression of the reporter polypeptide.

Additional control experiments may be carried out.

Suitable test substances which can be tested in the above assays include combinatorial libraries, defined chemical entities and compounds, peptide and peptide mimetics, oligonucleotides and natural product libraries, such as display (e.g. phage display libraries) and antibody products.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show inhibition or activation tested individually. Test substances may be used at a concentration of from 1 nM to 10 mM, preferably from, 100 nM to 1000 μM or from 1 M to 100 μM, more preferably from 1 μM to 10 μM. Preferably, the activity of a test substance is compared to the activity shown by a known activator or inhibitor. A test substance which acts as an inhibitor may produce a 50% inhibition of activity of the receptor. Alternatively a test substance which acts as an activator may produce 50% of the maximal activity produced using a known activator.

Another aspect of the present invention is the use of polynucleotides encoding the HIPHUM 119 polypeptides of the invention to identify mutations in HIPHUM 119 genes which may be implicated in human disorders. Identification of such mutations may be used to assist in diagnosis or susceptibility to such disorders and in assessing the physiology of such disorders. Polynucleotides may also be used in hybridisation studies to monitor for up- or down-regulation of HIPHUM 119 expression. Polynucleotides such as SEQ ID NO: 1 or fragments thereof may be used to identify allelic variants, genomic DNA and species variants.

The present invention provides a method for detecting variation in the expressed products encoded by HIPHUM 119 genes. This may comprise determining the level of an HIPHUM 119 expressed in cells or determining specific alterations in the expressed product. Sequences of interest for diagnostic purposes include, but are not limited to, the conserved portions as identified by sequence similarity and conservation of intron/exon structure. The diagnosis may be performed in conjunction with kindred studies to determine whether a mutation of interest co-segregates with disease phenotype in a family.

Diagnostic procedures may be performed on polynucleotides isolated from an individual or alternatively, may be performed in situ directly upon tissue sections (fixed and/or frozen) of patient tissue obtained from biopsies or resections, such that no nucleic acid purification is necessary. Appropriate procedures are described in, for example, Nuovo, G. J., 1992, “PCR In Situ Hybridization: Protocols And Applications”, Raven Press, NY). Such analysis techniques include, DNA or RNA blotting analyses, single stranded conformational polymorphism analyses, in situ hybridization assays, and polymerase chain reaction analyses. Such analyses may reveal both quantitative aspects of the expression pattern of a HIPHUM 119, and qualitative aspects of HIPHUM 119 expression and/or composition.

Alternative diagnostic methods for the detection of HIPHUM 119 nucleic acid molecules may involve their amplification, e.g. by PCR (the experimental embodiment set forth in U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. 15 USA 86:1173-1177), Q-Beta Replicase (Lizardi et al., 1988, Bio/Technology 6:1197) or any other nucleic acid amplification method, followed by the detection of the amplified molecules using techniques well known to those of skill in the art. These detection schemes are especially useful for the detection of nucleic acid molecules if such molecules are present in very low numbers.

Particularly suitable diagnostic methods are chip-based DNA technologies such as those described by Hacia et al., 1996, Nature Genetics 14:441-447 and Shoemaker et al., 1996, Nature Genetics 14:450-456. Briefly, these techniques involve quantitative methods for analyzing large numbers of nucleic acid sequence targets rapidly and accurately. By tagging with oligonucleotides or using fixed probe arrays, one can employ chip technology to segregate target molecules as high density arrays and screen these molecules on the basis of hybridization.

Following detection, the results seen in a given patient may be compared with a statistically significant reference group of normal patients and patients that have HIPHUM 119 related pathologies. In this way, it is possible to correlate the amount or kind of HIPHUM 119 encoded product detected with various clinical states or predisposition to clinical states.

Another aspect of the present invention is the use of the substances that have been identified by screening techniques referred to above in the treatment of disease states, which are responsive to regulation of cysteine proteinase activity. The treatment may be therapeutic or prophylactic. The condition of a patient suffering from such a disease state can thus be improved.

In particular, such substances may be used in the treatment of HIV infection, lung cancer, inflammatory disease such as asthma, Hepatitis B and brain diseases such as Alzheimer's disease, parasupranuclear palsey and Huntington's disease.

Additional disease states that may be treated include chronic obstructive pulmonary disease (COPD), breast cancer, neuronal ceroid lipofuscinosis, Badet-Biedl syndrome, multiple sclerosis and allergic encephalomyelitis.

Substances that act as inhibitors of HIPHUM 119 activity may be used in the treatment of disease states in which HIPHUM 119 expression is up-regulated such as stimulated bone marrow, HIV infection and HIV/PBL infection. Substances that act as activators of HIPHUM 119 activity may be used in the treatment of disease states in which expression of HIPHUM 119 is down-regulated such as brain diseases (Alzheimer's disease, parasupranuclear palsey, and Huntington's disease), lung tumors, lung asthma and Hepatitis B infection.

Substances identified according to the screening methods outlined above may be formulated with standard pharmaceutically acceptable carriers and/or excipients as is routine in the pharmaceutical art. For example, a suitable substance may be dissolved in physiological saline or water for injections. The exact nature of a formulation will depend upon several factors including the particular substance to be administered and the desired route of administration. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The substances may be administered by enteral or parenteral routes such as via oral, buccal, anal, pulmonary, intravenous, intra-arterial, intramuscular, intraperitoneal, topical or other appropriate administration routes.

A therapeutically effective amount of a modulator is administered to a patient. The dose of a modulator may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Nucleic acid encoding HIPHUM 119 or a variant thereof which inhibits HIPHUM 119 activity may be administered to the mammal. In particular, a nucleic acid encoding a polypeptide with HIPHUM 119 activity may be administered to a subject suffering from a condition in which HIPHUM 119 expression is down-regulated, such as Alzheimer's disease, parasupranuclear palsey, Huntington's disease, lung tumor, lung asthma and Hepatitis B infection. A nucleic acid encoding a variant of HIPHUM 119 that inhibits HIPHUM 119 activity may be administered to a patient suffering from a condition in which HIPHUM 119 expression is up-regulated such as HIV infection and HIV/PBL infection. Nucleic acid, such as RNA or DNA, and preferably, DNA, is provided in the form of a vector, such as the polynucleotides described above, which may be expressed in the cells of the mammal.

Nucleic acid encoding the polypeptide may be administered by any available technique. For example, the nucleic acid may be introduced by needle injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

The following Examples illustrate the invention.

### EXAMPLE 1

### Characterisation of the Sequence

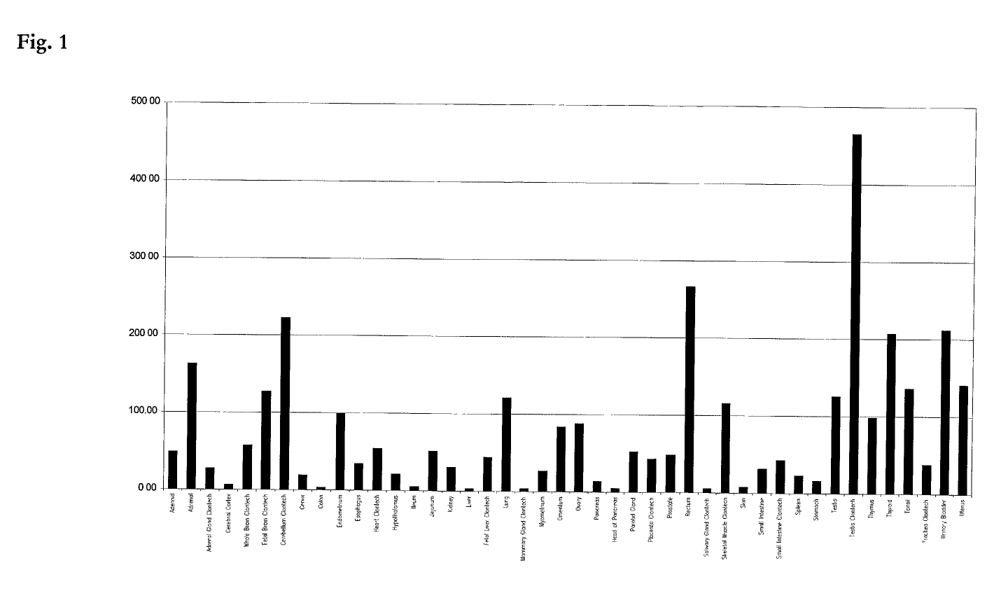

A cysteine proteinase, designated as HIPHUM 119 has been identified. The nucleotide and amino acid sequences of the receptor have been determined. These are set out below in SEQ ID NOs: 1 and 2. Suitable primers and probes were designed and used to analyse tissue expression. HIPHUM 119 was found to be expressed in all tissues at various levels (FIG. 1). It is highly expressed in adrenal, cerebellum, rectum, testis, thyroid and urinary bladder. It is also expressed at significant levels in lung, fetal brain, skeletal muscle, tonsil and uterus. In addition, it is expressed in T cells, peripheral blood mononucleocytes (PBMNCs), monocytes and dendritic cells.

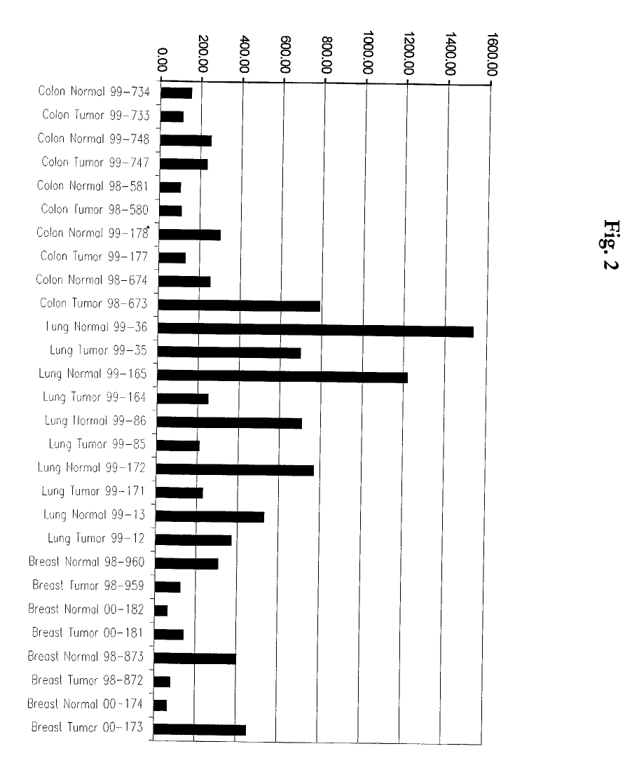

HIPPIHUM 119 expression levels are down regulated in lung tumors, lung asthma, and Hepatitis B infection (FIG. 2).

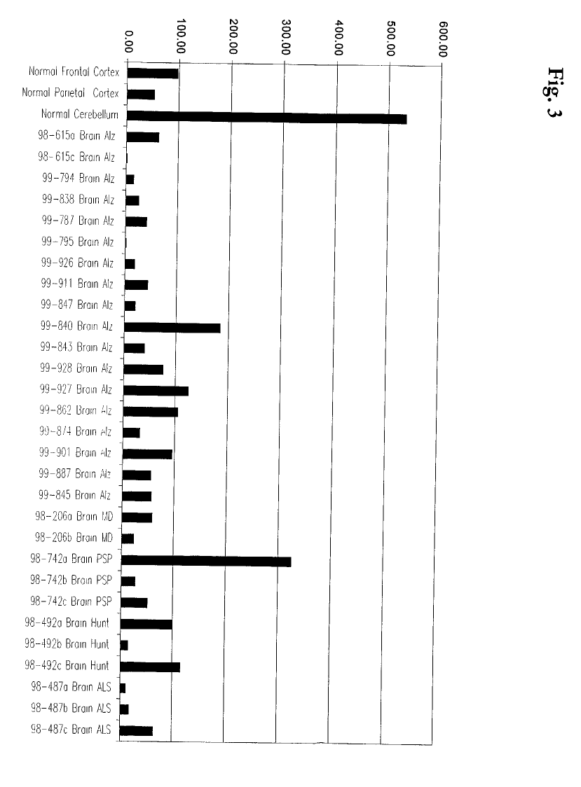

HIPPIHUM 119 expression levels are down regulated in Alzheimer's disease, parasupranuclear palsey, Huntington's disease, lung tumors, lung asthma, and Hepatitis B infection (FIG. 3).

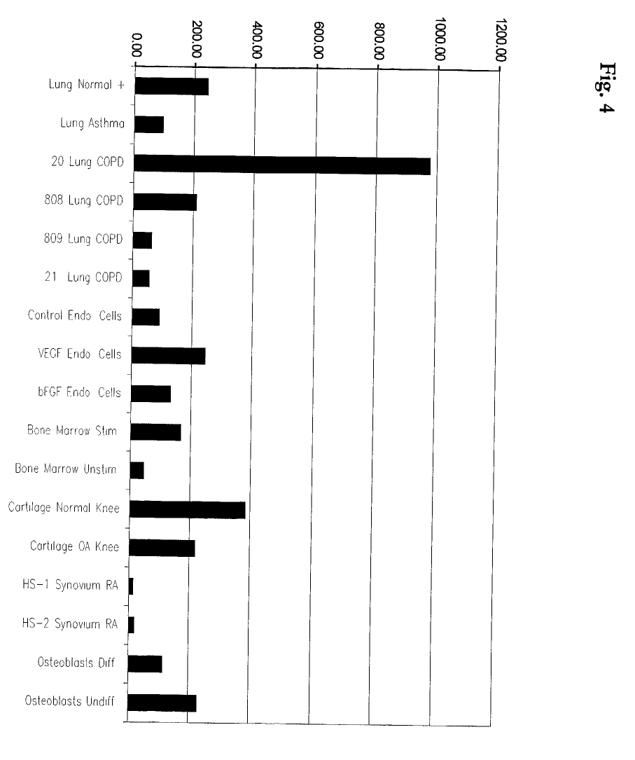

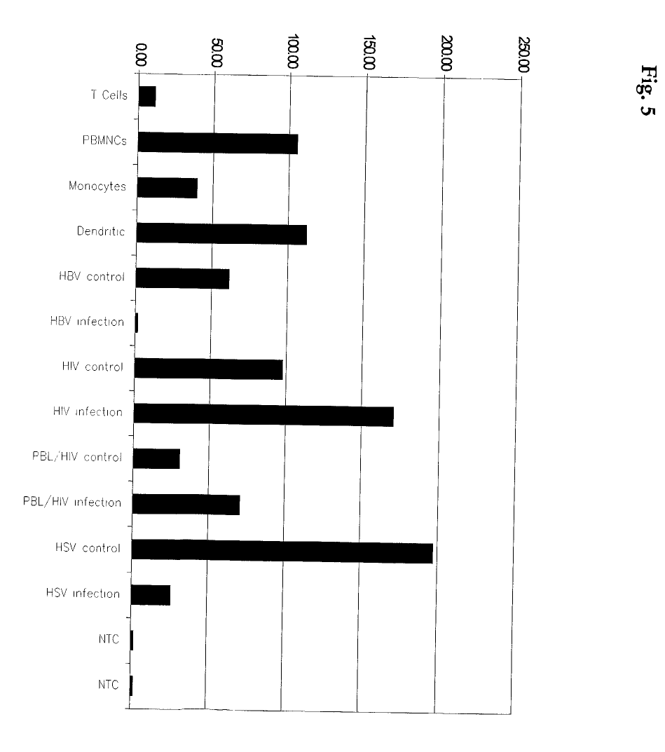

HIPPIHUM 119 expression levels are elevated in stimulated bone marrow (FIG. 4), HIV infection and HIV/PBL infection (FIG. 5).

The chromosomal localization was also mapped. Human HIPHUM 119 has been mapped to 15q22.

### EXAMPLE 2

### Screening for Substances Which Exhibit Protein Modulating Activity

Preparations of a purified polypeptide of the invention are generated for screening purposes. 96 and 384 well plate, high throughput screens (HTS) are employed using fluorescence or colourimetric indicator molecules. Secondary screening involves the same technology. Tertiary screens involve the study of modulators in rat, mouse and guinea-pig models of disease relevant to the target.

A brief screening assay protocol is as follows:

A polypeptide of the invention is expressed in E. coli, purified and refolded by direct dilution in assay buffer (200 mM NaCl, 50 mM Tris, 5 mM CaCl₂, 10 μM ZnSO₄, 0.01% Brij 35, pH 7.5). Test substances are provided in pools of 10 at 5 mM for each test substance for high throughput screening or are serially diluted in dose response assays. The screening assay is run on an automated system incorporating an OCRA rail to move the plates, a Tecan liquid handler, a Multimek liquid handler and a Titertek liquid handler. (The assay may also be run on manually or in combination with any suitable liquid handling equipment.)

60 μl of serially diluted test substance is added to 96 well plates (4 control wells with no inhibitor), 60 μl of a solution of a polypeptide of the invention (0.5 μM to 150 μM) is then added to each well and 50 mM EDTA (20 of 0.5M EDTA) is added to 4 inhibited control wells. In a fluorescence assay, the plates are read in a Fluostar (SLT) fluorescence plate reader or equivalent at an excitation of 343 nm and an emission of 450 nm. In a colourimetric assay the plates are read continuously in a SLT spectrophotometer at 405 nm for 3 minutes.

Percentage inhibition is calculated for each concentration (unknown values=U) in the dose response based on the range determined using the value of the difference of the control (no test substance) (mean of 4=C1) and the EDTA treated wells (mean of 4=C2) using the equation 100*(1−(U−C2)/(C1−C2).

1. An isolated cysteine proteinase polypeptide comprising
(i) the amino acid sequence of SEQ ID NO: 2; or
(ii) a variant thereof which is capable of cleaving SUMO from a target protein and/or cleaving the precursor form of SUMO to release the active form of SUMO; or
(iii) a fragment of (i) or (ii) which is capable of cleaving SUMO from a target protein and/or cleaving the precursor form of SUMO to release the active form of SUMO.

2. A polypeptide according to claim 1 wherein the variant (ii) has at least 80% identity to the amino acid sequence of SEQ ID NO: 2.

3. A polynucleotide encoding a polypeptide according to claim 1.

4. A polynucleotide according to claim 3 which is a cDNA sequence.

5. A polynucleotide encoding a cysteine proteinase polypeptide which is capable of cleaving SUMO from a target protein and/or cleaving the precursor form of SUMO to release the active form of SUMO which polynucleotide comprises:
(a) the nucleic acid sequence of SEQ ID NO: 1 and/or a sequence complementary thereto;
(b) a sequence which hybridises under stringent conditions to a sequence as defined in (a);
(c) a sequence that is degenerate as a result of the genetic code to a sequence as defined in (a) or (b); or
(d) a sequence having at least 60% identity to a sequence as defined in (a), (b) or (c).

6. An expression vector comprising a polynucleotide according to claim 5.

7. A host cell comprising an expression vector according to claim 6.

8. An antibody specific for a polypeptide according to claim 1.

9. A method for the identification of a substance that modulates cysteine proteinase activity, which method comprises:
(i) contacting a test substance and a polypeptide according to claim 1, and
(ii) determining the effect of the test substance on the activity of the said polypeptide, thereby to determine whether the test substance modulates cysteine proteinase activity.

10. A method according to claim 9 wherein the polypeptide is in a substantially isolated form.

11. A substance which modulates cysteine proteinase activity and which is identifiable by a method according to claim 9.

12. A method of treating a subject having a disorder that is responsive to cysteine proteinase modulation, which method comprises administering to said subject an effective amount of a substance according to claim 11.

13. A method according to claim 12 wherein the disorder is selected from HIV infection, lung cancer, inflammatory disease, Hepatitis B and brain disease.

14. A method of producing a polypeptide capable of cleaving SUMO from a target protein, which method comprises maintaining a host cell according to claim 7 under conditions suitable for obtaining expression of the polypeptide and isolating the said polypeptide.

15. A method for identification of a substance that modulates expression of a cysteine proteinase polypeptide, the method comprising:
(a) administering a test substance to a cell expressing a cysteine proteinase polypeptide according to claim 1, and
(b) determining the effect of the test substance on the expression of said polypeptide, thereby to determine whether the test substance modulates expression of said polypeptide.


SEQUENCE LISTING (2 sequences)

SEQ ID NO: 1 - DNA, 639 bp, Homo sapiens
  CDS (1)..(636)
atggaccccgtagtcttgagttacatggacagtctactgcggcaatcagatgtctcactattggatccgccaagctggctcaatgaccatattattgggtttgcgtttgagtactttgccaacagtcagtttcatgactgctctgatcacgtcagtttcatcagccctgaagtcacccagttcatcaagtgcactagcaacccagcagagattgccatgttccttgaaccactggacctccccaacaagagagttgtatttttagccatcaatgataactccaaccaggcagctggaggaacccactggagtttattggtctacctccaagataaaaatagcttttttcattatgattcccatagcaggagcaactcagttcacgcaaagcaggtagcagagaaactggaggctttcttaggcagaaaaggagacaaactggcctttgtggaagagaaagcccctgcccaacaaaacagctatgactgtgggatgtacgtgatatgtaacactgaggccttgtgtcagaacttctttaggcaacagacagaatcactgctgcagctactcacccctgcatacatcacaaagaagaggggagaatggaaagatctcattgccacacttgctaaaaagtag

SEQ ID NO: 2 - protein, 212 aa, Homo sapiens
MDPVVLSYMDSLLRQSDVSLLDPPSWLNDHIIGFAFEYFANSQFHDCSDHVSFISPEVTQFIKCTSNPAEIAMFLEPLDLPNKRVVFLAINDNSNQAAGGTHWSLLVYLQDKNSFFHYDSHSRSNSVHAKQVAEKLEAFLGRKGDKLAFVEEKAPAQQNSYDCGMYVICNTEALCQNFFRQQTESLLQLLTPAYITKKRGEWKDLIATLAKK